(19) United States
(12) Patent Application Publication
(10) Pub. No.: US 2025/0367068 A1
(43) Pub. Date: Dec. 4, 2025

(54) A VIBROACOUSTIC TACTILE DEVICE

(71) Applicant: Carl Simmons, Wootton Northamptonshire (GB)

(72) Inventor: Carl Simmons, Wootton Northamptonshire (GB)

(21) Appl. No.: 18/875,023

(22) PCT Filed: Jun. 15, 2023
(86) PCT No.: PCT/GB2023/051571

§ 371 Date: Dec. 13, 2024

(30) Foreign Application Priority Data
Jun. 15, 2022 (GB) 2208815.7

(51) Int. Cl. A61H 23/02; A61M 21/00; A61M 21/02

(52) U.S. Cl. CPC A61H 23/0236; A61H 23/0245; A61M 21/02; A61H 2201/0142; A61H 2201/0161; A61H 2205/02; A61H 2205/12; A61M 2021/0022; A61M 2021/0027

(57) ABSTRACT

A vibroacoustic tactile device and method of transmitting a vibroacoustic signal onto a human body includes at least one vibroacoustic tactile panel. The one or more vibroacoustic tactile panel has a vibroacoustic generating layer, a tactile surface, and a vibroacoustic transmission layer. The vibroacoustic transmission layer is disposed between the vibroacoustic generating layer and the tactile surface. The vibroacoustic tactile device further includes a signal delivery module that is operated to deliver an acoustic signal to the vibroacoustic generating layer. The vibroacoustic generating layer is operated to generate a vibroacoustic signal incident on the vibroacoustic transmission layer and the vibroacoustic transmission layer is operated to transmit the vibroacoustic signal to the tactile surface onto a human body.

### TECHNICAL FIELD

The present invention relates to a load bearing vibroacoustic tactile device and particularly a vibroacoustic tactile device for generating and delivering a vibroacoustic signal to be experienced audibly and directly through the felt sense of a human body.

### BACKGROUND

Vibroacoustic stimulation (VAS) involves transmitting low frequency vibrations into a human body. An example of an application of VAS is Vibroacoustic Therapy (VAT) which has been approved by the Food and Drug Administration (FDA), in United States of America, for increasing circulation, pain relief and increasing mobility. It is also been found to be an effective tool for encouraging relaxation and meditation and believed to have other therapeutic affects which are currently being researched. Additionally, the vibroacoustic effect has also been found to be an effective anchor for meditation.

Patent Document No. EP0244508A2 describes a device having a closed box with a sound opening in which is arranged a loudspeaker, of the coned type. The loudspeaker is directed towards a part of a human body lying on the closed box. Upholstery is disposed between the box and the human body in the location of the loudspeaker. Low frequency signals are supplied to the loudspeaker. The device also has external loudspeakers for playing music. The low frequency signals are influenced in step with the music.

Known vibroacoustic therapy devices, such as described in EP0244508A2, are disadvantaged in that they typically use coned audio speakers to transmit at a limited band of low frequency signal and directionally onto an isolated part of the human body. Such devices are also disadvantaged in that they also have relatively large, heavy, and cumbersome structures which are not of a portable structure and have a limited utility, solely for use as vibroacoustic therapy devices.

It is therefore desirable in the industry for there to be a relatively inexpensive, lightweight and portable vibroacoustic stimulation (VAS) device which is capable of transmitting low frequency signals uniformly to the whole of the human body for therapeutic uses and a full range of frequencies fir wider applications such as leisure, relaxation and general wellbeing.

### SUMMARY OF INVENTION

According to a first aspect of the present invention, there is provided a vibroacoustic tactile device comprising at least one vibroacoustic tactile panel, the, or each, vibroacoustic tactile panel comprising a vibroacoustic generating layer, a tactile surface, and a vibroacoustic transmission layer, wherein the vibroacoustic transmission layer is disposed between the vibroacoustic generating layer and the tactile surface, and wherein the vibroacoustic tactile device further comprises a signal delivery module operable to deliver an acoustic signal to the vibroacoustic generating layer and wherein the vibroacoustic generating layer is operable to generate a vibroacoustic signal incident on the vibroacoustic transmission layer and the vibroacoustic transmission layer is operable to transmit the vibroacoustic signal to the tactile surface.

The vibroacoustic generating layer advantageously comprises a plurality of supports for supporting the vibroacoustic transmission layer and arranged to advantageously define regions of enclosed space within the vibroacoustic generating layer and underlying the vibroacoustic transmission layer.

The vibroacoustic generating layer advantageously comprises a closed foam material. The closed foam material may comprise Ethylene-vinyl acetate (EVA). Alternatively, or additionally, the closed foam material may comprise Expanded Polypropylene (EPP). The closed foam material may be formed of a suitable density to support the vibroacoustic transmission layer but also provide sufficient movement to not inhibit free movement of the vibroacoustic transmission layer.

The vibroacoustic tactile device advantageously further comprises one or more vibroacoustic tactile transducers disposed in the vibroacoustic generating layer and operable to receive an acoustic signal from the signal delivery module, generate a vibroacoustic signal, and transmit the generated vibroacoustic signal onto the vibroacoustic transmission layer. The one or more vibroacoustic tactile transducers is advantageously operable within the frequency range of 20 Hz to 200 Hz.

The vibroacoustic tactile device advantageously further comprises at least one vibroacoustic exciter, wherein the at least one vibroacoustic exciter is disposed in the vibroacoustic generating layer and advantageously operable to receive an acoustic signal from the signal delivery module, generate a vibroacoustic signal and transmit the generated vibroacoustic signal onto the vibroacoustic transmission layer. The at least one vibroacoustic exciter is advantageously operable within the frequency range 200 Hz to 20 KHz.

The signal delivery module advantageously comprises a signal amplifier.

The vibroacoustic transmission layer advantageously comprises a soundboard formed from a wooden material.

The tactile surface is advantageously formed from a Thermo Elastomer (TPE) material or other similar material capable of providing a similar function.

The vibroacoustic tactile device advantageously comprises a plurality of vibroacoustic tactile panels. Each vibroacoustic tactile panel is advantageously detachably attachable to an adjacent panel and may be arrangeable relative to each other depending on the application, such as, for example, therapy, meditation, fitness, lounger chair and wall panels.

Each vibroacoustic tactile panel is advantageously hingedly attached to an adjacent one of the plurality of vibroacoustic tactile panels.

The vibroacoustic tactile device is advantageously operable to support the body of a human and transmit a vibroacoustic signal onto a said human body through the tactile surface.

The one or more vibroacoustic tactile transducers is advantageously disposed at predetermined regions of the vibroacoustic transmission layer to cause said vibroacoustic signal to be more concentrated at one or more regions of the tactile surface corresponding with predetermined portions of the body of said human supported on the tactile surface.

One or more vibroacoustic exciters is advantageously disposed at a predetermined region of the vibroacoustic transmission layer to cause said vibroacoustic signal to be more concentrated at one or more regions of the tactile surface corresponding with the head of the body of said human supported on the tactile surface.

According to a second aspect of the present invention there is provided vibroacoustic tactile system comprising a plurality of vibroacoustic tactile devices according to the first aspect of the present invention.

The plurality of vibroacoustic tactile devices are advantageously responsive to a common vibration signal.

The plurality of vibroacoustic tactile devices are advantageously remotely controlled.

According to a third aspect of the present invention there is provided a method of transmitting a vibroacoustic signal onto a human body, the method comprising: providing a vibroacoustic tactile device, according to the first aspect of the present invention; disposing at least part of said human body on the tactile surface; providing an acoustic signal to the signal delivery module; actuating the signal delivery module to generate an acoustic signal in the vibroacoustic generating layer; transducing the acoustic signal into a vibroacoustic signal incident on the vibroacoustic transmission layer; and transmitting the vibroacoustic signal to the tactile surface.

### BRIEF DESCRIPTION OF DRAWINGS

Various embodiments of the invention will be described with reference to the following drawings, in which:

FIG. 1 is a drawing showing a vibroacoustic tactile device according to a first embodiment of the present invention;

FIG. 2 is a drawing showing an alternative embodiment of a vibroacoustic tactile device according to the present invention, shown in a folded state;

FIG. 3 is a drawing showing a vibroacoustic generating layer of the vibroacoustic tactile device of FIG. 2, shown from above;

FIG. 4 is a drawing showing a section through the vibroacoustic generating layer of FIG. 3;

FIG. 5 is a drawing showing an exploded view of the vibroacoustic tactile device of FIG. 1;

FIG. 6 is a drawing showing the exploded view of the vibroacoustic tactile device of FIG. 5, having a signal delivery module, vibroacoustic tactile transducers and vibroacoustic exciters disposed in the vibroacoustic generating layer;

The present invention will be described more fully hereinafter with reference to the accompanying drawings. Like numbers refer to like elements throughout. The vibroacoustic tactile device and parts thereof are not necessarily to scale and the parts may just be representative of components or other described entities.

### DESCRIPTION OF EMBODIMENTS

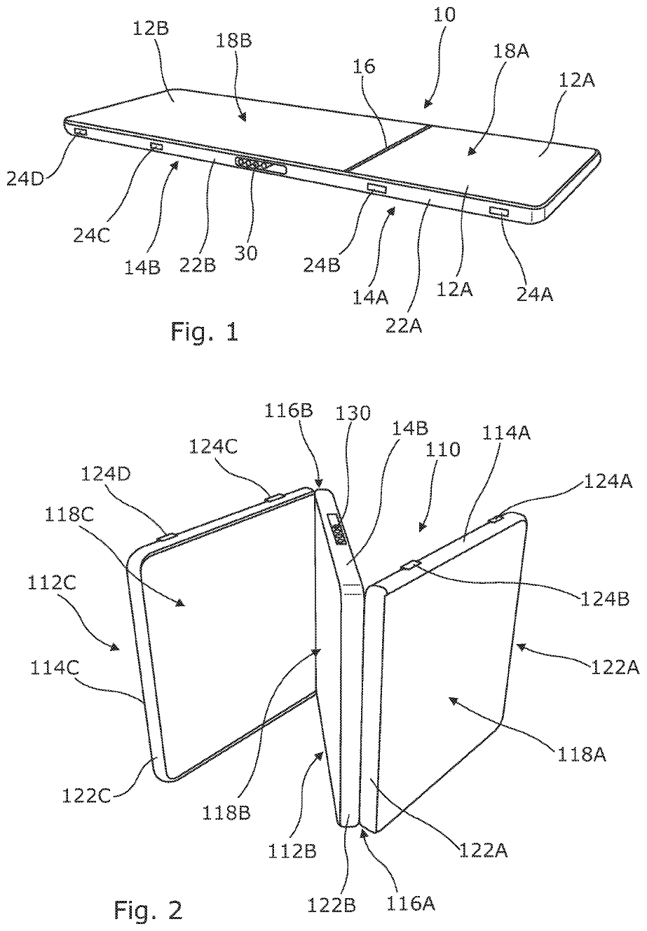

Referring to FIG. 1, according to an embodiment of the present invention a vibroacoustic tactile device 10 comprises a first vibroacoustic tactile panel, 12A and a second vibroacoustic tactile panel 12B. Each vibroacoustic tactile panel, 12A and 12B, comprise a base layer, 14A and 14B, and a top layer, 18A and 18B, which functions to provide tactile surfaces 20A and 20B. The base layers, 14A and 14B, comprise durable housings 22A and 22B formed from, for example, a plastics material. The vibroacoustic tactile panels, 12A and 12B are attached to each other in a hinged arrangement 16 and can be folded relative to each other. In this embodiment, in which there are two vibroacoustic tactile panels, 12A and 12B, the first vibroacoustic tactile panel 12A has a relatively larger surface area of tactile surface 20A and the second vibroacoustic tactile panel 12B has a relatively smaller surface area of tactile surface 20B.

When used as a support or bed for a human body, the vibroacoustic tactile panel 12A having a relatively larger surface area of tactile surface 20A is operable to support the body of a human and the second vibroacoustic tactile panel 12B, having a relatively smaller surface area of tactile surface 20B, is operable to support the head of a human. Each panel may have an approximately equal weight-bearing capability but may differ in the provision of low or higher frequency effects dependent on the type of vibroacoustic exciters and vibroacoustic tactile transducers utilised in the vibroacoustic tactile device 10.

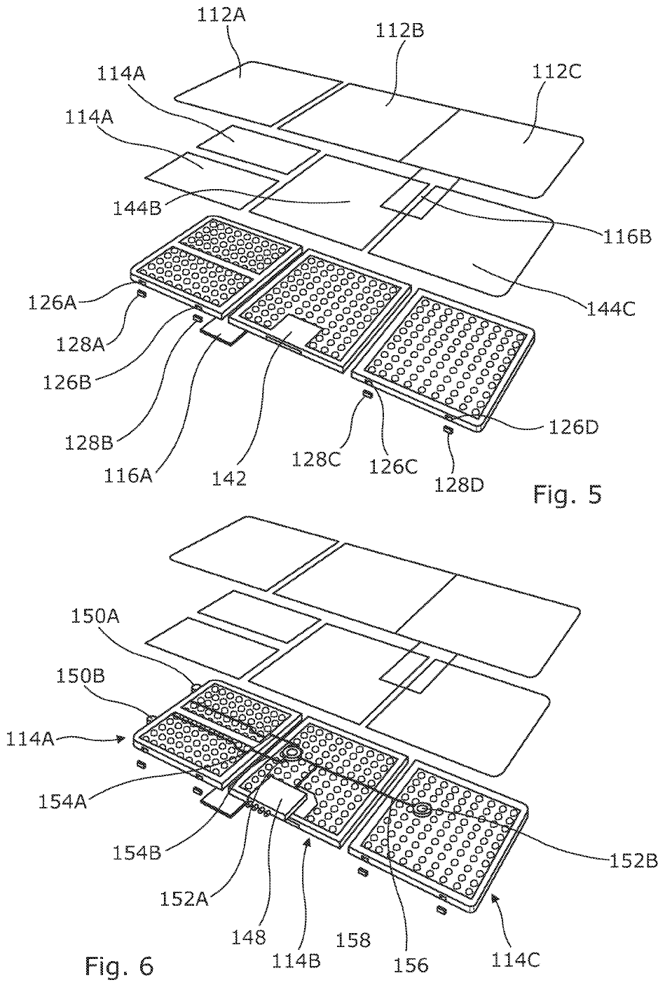

The housings, 22A and 22B, comprise a plurality of carrying handles, shown as 24A, 24B, 24C and 24D, on one side of the housing 22. The opposing side of the housing 22 has corresponding handles but are not shown in FIG. 1. Alternative embodiments of the present invention may have different numbers of handles arranged on the housing in different configurations. Referring to FIG. 5, the handles 24A, 24B, 24C and 24D are formed as sockets 26A, 26B, 26C and 26D in the wall of the housing 22. Corresponding handle plugs 28A, 28B, 28C and 28D are dimensioned and shaped to provide a friction fit with the corresponding sockets 26A, 26B, 26C and 26D. With the handle plugs 28A, 28B, 28C and 28D inserted in the sockets 26A, 26B, 26C and 26D the external surface of the wall of the housing is substantially uninterrupted and planar.

Disposed in the base layer is a signal delivery module 30 having an amplifier operable to receive an audio signal.

In an alternative embodiment (not shown in the drawings), the vibroacoustic tactile device may comprise a single vibroacoustic tactile panel. The single vibroacoustic tactile panel having a base layer and a top layer 18, which functions to provide a tactile surface 20. The single vibroacoustic tactile panel has similar features and operates in the same way as the vibroacoustic tactile device 10, of FIG. 1, except it cannot be folded.

Referring to FIG. 2, an alternative embodiment of a vibroacoustic tactile device 110, according to the present invention, is similar to the embodiment of FIG. 1. However, the embodiment of FIG. 2 comprises three vibroacoustic tactile panels, 112A, 112B and 112C. Each vibroacoustic tactile panel, 112A, 112B and 112C, comprises a base layer, 114A, 114B and 114C, and a top layer 118A, 118B and 118C, which function to provide a tactile surfaces 120A, 120B and 120C. Each of the base layers, 114A, 114B and 114C, comprise durable housings, 122A, 122B and 122C, formed from, for example, a plastics material. The vibroacoustic tactile panels, 112A, 112B and 112C are attached in series in a hinged arrangements 116A and 116B.

The top layers 118A, 118B and 118C may be formed from, for example, a Thermo Elastomer (TPE) material or similar material.

A plurality of carrying handles, shown as 124A, 124B, 124C and 124D, on one side of the housings 122A, 122B and 122C. The opposing side of the housings, 122A, 122B and 122C, have corresponding handles but are not shown in FIG. 2. Alternative embodiments of the present invention may have different numbers of handles arranged on the housing in different configurations. The handles 124A, 124B, 124C and 24D are the same as those described in relation to the first embodiment, with reference to FIG. 1.

An audio signal module 130 having an amplifier operable to receive an audio signal is disposed in one of the vibroacoustic tactile panel, 112A, 112B and 112C.

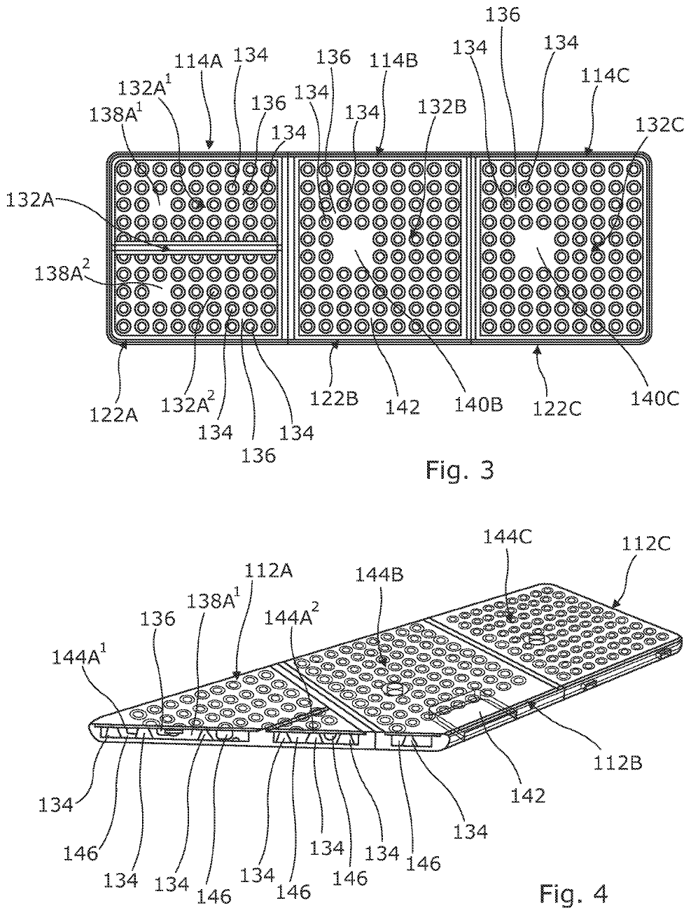

Referring also to FIG. 3 the base layers, 114A, 114B and 114C, of each of the vibroacoustic tactile panels, 112A, 112B and 112C, comprise the housings 122A, 122B and 122C. Each of the housings 122A, 122B and 122C has a cavity in which vibroacoustic generating layers, 132A, 132B and 132C, are disposed respectively. One of the vibroacoustic generating layers may be divided into two separate sub-vibroacoustic generating layers 132A¹ and 132A², disposed to lie next to each other in the housing 122A.

The vibroacoustic generating layers, 132A¹ and 132A², 132B and 132C, are formed from a closed foam material, such as, for example, Ethylene-vinyl acetate (EVA).

Each of the vibroacoustic generating layers, 132A¹ and 132A², 132B and 132C, has a plurality of supports 134 spaced apart relative to each other to define regions of space 136 when the vibroacoustic tactile device is in an assembled state.

Each of the vibroacoustic generating layers 132A¹ and 132A² further comprise vibroacoustic exciter housing spaces 138A¹ and 138A², respectively, formed within the supports 134. Each of the vibroacoustic generating layers 132B and 132C further comprise a vibroacoustic tactile transducer housing spaces 140B and 140C, respectively, formed within the supports 134.

Vibroacoustic generating layer 132B additionally comprises a signal delivery module housing space 142 formed within the supports 134.

Referring also to FIGS. 4, 5 and 6, vibroacoustic transmission layers 144A¹, 144A², 144B and 144C are disposed to overlie the vibroacoustic generating layers 132A¹, 132A², 132B and 132C, respectively, supported on the supports 134 to form enclosed vibroacoustic transmission spaces 146. The vibroacoustic transmission layers 144A¹, 144A², 144B and 144C may be formed of a wooden material, such as, for example, plywood sheets which function as a sound/vibroacoustic board. The vibroacoustic transmission layers 144A¹, 144A², 144B and 144C are preferably arranged and operable to float on the vibroacoustic generating layers 132A¹, 132A², 132B and 132C, respectively, and act as vibroacoustic floating supports layers.

Suitable material is attached to adjacent vibroacoustic tactile panels, 112A, 112B and 112C and function as hinges 116A and 116B.

Referring in particular to FIGS. 5 and 6, a signal delivery module 148 is disposed in the signal delivery module housing space 142 of the vibroacoustic generating layer 132B. The signal delivery module 148 preferably comprises an amplifier.

Vibroacoustic exciters, 150A and 150B are disposed in the housing spaces 138A¹ and 138A², respectively.

Vibroacoustic tactile transducers, 152A and 152B, are disposed in the vibroacoustic tactile transducer housing spaces 140B and 140C, respectively.

The vibroacoustic exciters, 150A and 150B, are electrically connected to vibroacoustic tactile transducers 152A, through electrical connections, 154A and 154B, respectively.

Vibroacoustic tactile transducer 152A is electrically connected to vibroacoustic tactile transducers 152B through electrical connection 156.

The vibroacoustic tactile transducers, 152A and 152B, are electrically connected to signal delivery module 148 through electrical connection 158 which is connected to electrical connection 156 at a T-junction.

The vibroacoustic exciters, 150A and 150B, are preferable operable within the frequency range 200 Hz to 20 KHz.

The vibroacoustic tactile transducers, 152A and 152B, are preferably operable within the frequency range of 20 Hz to 200 Hz.

It will be appreciated by the skilled person in the art that the components of the vibroacoustic tactile device, as described herein, may be differently arranged and/or formed within the scope of the present invention, as claimed.

In use, the signal delivery module 148 is activated to generate an acoustic signal. The acoustic signal is transmitted from the signal delivery module 148 through the electrical connections 158 and 156, to activate the vibroacoustic tactile transducers, 152A and 152B, which transmit a tactile vibroacoustic signal in the vibroacoustic generating layers, 132B and 132C, respectively, within the frequency range 20 Hz to 200 Hz. The tactile vibroacoustic transducer generated signal is dissipated from the vibroacoustic tactile transducers, 152A and 152B, through the enclosed vibroacoustic transmission space 146, to provide a uniformly distributed tactile vibroacoustic signal throughout the vibroacoustic generating layers, 132B and 132C, respectively.

The uniformly distributed tactile vibroacoustic signal in the enclosed vibroacoustic transmission space 146 is incident uniformly on the vibroacoustic transmission layers 144B and 144C. The uniformly distributed tactile vibroacoustic signal is transmitted through the vibroacoustic transmission layers 144B and 144C and onto the tactile surfaces 120B and 120C, respectively, such that the uniformly distributed tactile vibroacoustic signal can be felt on the tactile surfaces 120B and 120C, at a frequency within the range of 20 Hz to 200 Hz.

Similarly, the acoustic signal, transmitted from the signal delivery module 148, is transmitted to the vibroacoustic exciters, 150A and 150B, via vibroacoustic tactile transducer 152A, through electrical connections 154A and 154B. The vibroacoustic exciters, 150A and 150B, generate a vibroacoustic signal within the frequency range of 200 Hz to 20 KHz. The vibroacoustic exciter generated signal is dissipated from the vibroacoustic exciters, 150A and 150B, through the enclosed vibroacoustic transmission space 146, to provide a uniformly distributed vibroacoustic exciter generated signal throughout the vibroacoustic generating layers, 132A¹ and 132A², respectively.

The uniformly distributed vibroacoustic exciter generated signal in the enclosed vibroacoustic transmission space 146 is incident uniformly on the vibroacoustic transmission layers 144A¹ and 144A². The uniformly distributed vibroacoustic exciter generated signal is transmitted through the vibroacoustic transmission layers 144A¹ and 144A² and onto the tactile surface 120A such that the uniformly distributed vibroacoustic exciter generated signal can be felt and/or heard on the tactile surfaces 120A, at a frequency within the range of 200 Hz to 20 KHz.

In use, the vibroacoustic tactile device 10/110 can be used as a therapy bed in which a human user assumes a horizontal or resting position on the top layer 118A, 118B and 118C, which function to provide the tactile surfaces 120A, 120B and 120C, respectively. The signal delivery module 148 delivers a signal into the vibroacoustic generating layers 132A¹, 132A², 132B and 132C, as described above. With the user's body disposed on the three vibroacoustic tactile panels, 112A 112B and 112C, the user experiences the uniformly distributed tactile vibroacoustic signal, at a frequency within the range of 20 Hz to 200 Hz, uniformly on their body. In this position, the user also experiences the uniformly distributed vibroacoustic exciter generated signal as a felt and/or heard sensation, at a frequency within the range of 200 Hz to 20 KHz, as the signal is transmitted into the tactile surface 120A around the head or feet of the user, depending on their orientation.

In an additional and/or alternative use, the vibroacoustic tactile device 10/110 functions as a good quality audio speaker and can be stood in an upright position by positioning the three vibroacoustic tactile panels, 112A 112B and 112C, at a hinged angle relative to each other, as shown in FIG. 2.

1. A vibroacoustic tactile device comprising at least one vibroacoustic tactile panel, the, or each, vibroacoustic tactile panel comprising a vibroacoustic generating layer, a tactile surface, and a vibroacoustic transmission layer, wherein the vibroacoustic transmission layer is disposed between the vibroacoustic generating layer and the tactile surface, and
wherein the vibroacoustic tactile device further comprises a signal delivery module operable to deliver an acoustic signal to the vibroacoustic generating layer and wherein the vibroacoustic generating layer is operable to generate a vibroacoustic signal incident on the vibroacoustic transmission layer and the vibroacoustic transmission layer is operable to transmit the vibroacoustic signal to the tactile surface.

2. The vibroacoustic tactile device as claimed in claim 1, wherein the vibroacoustic generating layer comprises a plurality of supports for supporting the vibroacoustic transmission layer and arranged to define regions of enclosed space within the vibroacoustic generating layer and underlying the vibroacoustic transmission layer.

3. The vibroacoustic tactile device as claimed in claim 1, wherein the vibroacoustic generating layer comprises a closed foam material.

4. The vibroacoustic tactile device as claimed in claim 3, wherein the closed foam material comprises ethylene-vinyl acetate.

5. The vibroacoustic tactile device as claimed in claim 1, further comprising one or more vibroacoustic tactile transducers disposed in the vibroacoustic generating layer and operable to receive an acoustic signal from the signal delivery module, generate a vibroacoustic signal, and transmit the generated vibroacoustic signal onto the vibroacoustic transmission layer.

6. The vibroacoustic tactile device as claimed in claim 5, wherein the one or more vibroacoustic tactile transducers is operable within the frequency range of 20 Hz to 200 Hz.

7. The vibroacoustic tactile device as claimed in claim 1, further comprising at least one vibroacoustic exciter, wherein the at least one vibroacoustic exciter is disposed in the vibroacoustic generating layer and operable to receive an acoustic signal from the signal delivery module, generate a vibroacoustic signal and transmit the generated vibroacoustic signal onto the vibroacoustic transmission layer.

8. The vibroacoustic tactile device as claimed in claim 7, wherein the at least one vibroacoustic exciter is operable within the frequency range 200 Hz to 20 KHz.

9. The vibroacoustic tactile device as claimed claim 1, wherein the signal delivery module comprises a signal amplifier.

10. The vibroacoustic tactile device as claimed in claim 1 wherein the vibroacoustic transmission layer comprises a sound/vibroacoustic board formed from a wooden material.

11. The vibroacoustic tactile device as claimed in claim 1, wherein the tactile surface is formed from a thermos elastomer material.

12. The vibroacoustic tactile device as claimed in claim 1, wherein the device comprises a plurality of vibroacoustic tactile panels.

13. The vibroacoustic tactile device as claimed in claim 12, wherein each panel is detachably attachable to an adjacent panel.

14. The vibroacoustic tactile device as claimed in claim 12, wherein each panel is hingedly attached to an adjacent one of the plurality of vibroacoustic tactile panels.

15. The vibroacoustic tactile device as claimed in claim 1, operable to support the body of a human and transmit a vibroacoustic signal onto a said human body through the tactile surface.

16. The vibroacoustic tactile device as claimed in claim 15, wherein the one or more vibroacoustic tactile transducers is disposed at predetermined regions of the vibroacoustic transmission layer to cause said vibroacoustic signal to be more concentrated at one or more regions of the tactile surface corresponding with predetermined portions of the body of said human supported on the tactile surface.

17. The vibroacoustic tactile device as claimed in claim 15, wherein one or more vibroacoustic exciters is disposed at a predetermined region of the vibroacoustic transmission layer to cause said vibroacoustic signal to be more concentrated at one or more regions of the tactile surface corresponding with the head of the body of said human supported on the tactile surface.

18-20. (canceled)

21. A method of transmitting a vibroacoustic signal onto a human body, comprising the steps of:
providing a vibroacoustic tactile device comprising at least one vibroacoustic tactile panel, the, or each, vibroacoustic tactile panel comprising a vibroacoustic generating layer, a tactile surface, and a vibroacoustic transmission layer, wherein the vibroacoustic transmission layer is disposed between the vibroacoustic generating layer and the tactile surface, and wherein the vibroacoustic tactile device further comprises a signal delivery module operable to deliver an acoustic signal to the vibroacoustic generating layer and wherein the vibroacoustic generating layer is operable to generate a vibroacoustic signal incident on the vibroacoustic transmission layer and the vibroacoustic transmission layer is operable to transmit the vibroacoustic signal to the tactile surface;
disposing at least part of said human body on the tactile surface;
providing an acoustic signal to the signal delivery module;
actuating the signal delivery module to generate an acoustic signal in the vibroacoustic generating layer;
transducing the acoustic signal into a vibroacoustic signal incident on the vibroacoustic transmission layer; and
transmitting the vibroacoustic signal to the tactile surface.
